(19) United States
(12) Patent Application Publication
(10) Pub. No.: US 2026/0027003 A1
(43) Pub. Date: Jan. 29, 2026

(54) FLEXIBLE NECK MASSAGER

(71) Applicant: Shenzhen Meilan Technology Co., Ltd., Shenzhen (CN)

(72) Inventor: Shaoqiao Zhang, Shenzhen (CN)

(21) Appl. No.: 19/345,408

(22) Filed: Sep. 30, 2025

(30) Foreign Application Priority Data
Apr. 23, 2025 (CN) 2025207855729

(51) Int. Cl. A61H 23/02; A61N 1/04; A61N 1/36

(52) U.S. Cl. CPC A61H 23/02; A61N 1/0404; A61N 1/36034; A61H 2201/0207; A61H 2205/04

(57) ABSTRACT

The utility model provides a flexible neck massager, which comprises a housing and a plurality of massage heads, wherein the housing comprises a support body and two holding arms; the support body is a flexible support body made of flexible materials, at least one metal strip is arranged in the flexible support body, and the two holding arms are respectively arranged at both ends of the flexible support body; the plurality of massage heads are fixed on the inner side of the flexible support body; a first accommodating cavity is arranged in the flexible support body, a first control circuit board is arranged in the first accommodating cavity, and the plurality of massage heads are electrically connected to the first control circuit board. The flexible materials can adapt to different neck shapes of the human bodies; moreover, the independently designed holding arms facilitate the assembly and disassembly of components.

### TECHNICAL FIELD

The utility model relates to the technical field of massage equipment, particularly to a flexible neck massager.

### BACKGROUND ART

With the advancement of modern information technology and the widespread popularization of smartphones, the number of people who spend long hours hunched over desks or looking down at their devices has significantly increased. While smartphones have revolutionized daily life, they have also posed threats to people's health. In recent years, cervical spondylosis has shown a younger trend. Those who spend prolonged periods looking down at their phones, books or computers, maintaining their head and neck in a single posture for extended durations, are particularly susceptible to developing cervical spondylosis.

Neck massagers can alleviate neck fatigue, promote neck blood circulation, and offer effective prevention against cervical spondylosis. Their simple and convenient operation has made them increasingly popular among consumers. Existing neck massagers typically consist of the support body and the massage heads which are often fixed to the support body using screws. However, since the housings of most current neck massagers are made of rigid materials like plastics, their elasticity and flexibility are limited. As a result, they often fail to adapt to the neck shapes of different individuals, leading to discomfort during wear.

In summary, it is evident that the prior art presents practical inconveniences and limitations, thus necessitating improvements.

SUMMARY OF THE UTILITY MODEL

In view of the above-mentioned defects, the utility model aims to provide a flexible neck massager that not only adapts to the neck shapes of different individuals for enhanced wearing comfort but also maintains structural rigidity while delivering effective holding and fixing effects.

To solve the above technical problems, the utility model is implemented as follows:

The utility model provides a flexible neck massager, comprising a housing and a plurality of massage heads, wherein the housing comprises a support body and two holding arms; the support body is a flexible support body made of flexible materials, and at least one metal strip is arranged in the flexible support body; the two holding arms are respectively arranged at both ends of the flexible support body; the plurality of massage heads are fixed on the inner side of the flexible support body; a first accommodating cavity is arranged in the flexible support body, a first control circuit board is arranged in the first accommodating cavity, and the plurality of massage heads are electrically connected to the first control circuit board.

According to the flexible neck massager of the utility model, the metal strip is embedded in the flexible support body during injection molding; or
the flexible support body is provided with at least one accommodating channel, and the metal strip is inserted into the accommodating channel.

According to the flexible neck massager of the utility model, the metal strip is made of common metal or memory metal.

According to the flexible neck massager of the utility model, the flexible support body is provided with a first fixing groove in the first accommodating cavity adapted to the shape of the first control circuit board, and the first control circuit board is fixed in the first fixing groove; and/or

The flexible support body is provided with at least one first opening at the position of the first accommodating cavity, the first opening is provided with a first sealing strip, and the first opening is detachably sealed through the first sealing strip; and/or
a plurality of mounting holes for mounting the plurality of massage heads are provided on the inner side of the flexible support body; and/or
at least one reinforcing rib is arranged in the first accommodating cavity of the flexible support body; and/or
a first electronic unit is also arranged on the flexible support body or in the first accommodating cavity and electrically connected to the first control circuit board; and/or
the holding arm is a hard holding arm made of hard materials.

According to the flexible neck massager of the utility model, each of the massage heads comprises a massage head housing and electrodes, the massage head housing is provided with a massage head accommodating cavity, and the electrodes are fixed at the top of the massage head housing and electrically connected to the first control circuit board.

According to the flexible neck massager of the utility model, the first control circuit board comprises a pulse drive circuit, which is electrically connected to the electrodes of the plurality of massage heads, and the electrodes output pulse current according to the pulse control instructions of the pulse drive circuit; and/or
a vibrating motor is arranged in the massage head accommodating cavity of the massage head housing; the first control circuit board comprises a motor drive circuit, which is electrically connected to the vibrating motors of the plurality of massage heads, and the vibrating motors vibrate according to the vibration control instructions of the motor drive circuit; and/or
a heating component is arranged in the massage head accommodating cavity of the massage head housing; the first control circuit board comprises a heating drive circuit, which is electrically connected to the heating components of the plurality of massage heads; the heating components heat up according to the heating control instructions of the heating drive circuit.

According to the flexible neck massager of the utility model, the holding arm comprises a first holding arm and a second holding arm;
a second accommodating cavity is arranged in the first holding arm, at least one second electronic unit is arranged on the first holding arm or in the second accommodating cavity, and a second control circuit board is arranged in the second accommodating cavity and electrically connected to the second electronic unit and the first control circuit board; and/or
a third accommodating cavity is arranged in the second holding arm, at least one third electronic unit is arranged on the second holding arm or in the third accommodating cavity, and a third control circuit board is arranged in the third accommodating cavity and electrically connected to the third electronic unit and the first control circuit board.

According to the flexible neck massager of the utility model, the first holding arm is composed of a first holding arm inner housing and a first holding arm outer housing;
the second holding arm is composed of a second holding arm inner housing and a second holding arm outer housing.

According to the flexible neck massager of the utility model, a second fixing groove adapted to the shape of the second control circuit board is arranged in the second accommodating cavity of the first holding arm, and the second control circuit board is fixed in the second fixing groove; and/or
a third fixing groove adapted to the shape of the third control circuit board is arranged in the third accommodating cavity of the second holding arm, and the third control circuit board is fixed in the third fixing groove.

According to the flexible neck massager of the utility model, a first flexible structure is arranged on the inner side of the first holding arm; and/or
a second flexible structure is arranged on the inner side of the second holding arm. The utility model provides a flexible neck massager, which comprises a housing and a plurality of massage heads, wherein the housing comprises a flexible support body and two holding arms; the plurality of massage heads are fixed on the inner side of the flexible support body; a first accommodating cavity is arranged in the flexible support body, a first control circuit board is arranged in first accommodating cavity, and he plurality of massage heads are electrically connected to the first control circuit board. Thus, the support body of the flexible neck massager in the utility model is made of flexible materials, allowing it to adapt to the neck shapes of different individuals, providing greater comfort during wear and enhancing the massage effect. Additionally, the metal strip is arranged in the flexible support body, ensuring structural rigidity. Moreover, the independently designed holding arms facilitate the assembly and disassembly of components.

### BRIEF DESCRIPTION OF THE DRAWINGS

FIG. 1 is a three-dimensional view of the flexible neck massager provided in Embodiment 1 of the utility model.

FIG. 2 is a sectional view of the flexible neck massager provided in Embodiment 1 of the utility model.

FIG. 3 is a three-dimensional exploded view of the flexible neck massager provided in Embodiment 1 of the utility model.

FIG. 4 is a circuit diagram of the flexible neck massager provided in Embodiment 2 of the utility model.

### DETAILED DESCRIPTION OF THE UTILITY MODEL

To provide a clearer understanding of the objectives, technical proposals and advantages of the utility model, the following offers a more detailed description with reference to the drawings and embodiments. It should be understood that the specific embodiments described herein are intended only to explain the utility model but not to limit it.

It should be noted that references in this specification to “one embodiment”, “an embodiment”, “an exemplary embodiment”, etc. mean that the described embodiment may include specific features, structures or characteristics. However, not every embodiment necessarily contains these specific features, structures or characteristics. Moreover, such expressions do not necessarily refer to the same embodiment. Furthermore, when specific features, structures or characteristics are described in connection with an embodiment, it has indicated that it is within the knowledge of those skilled in the art to incorporate such features, structures or characteristics into other embodiments, whether explicitly described or not.

Moreover, certain terms are used in the specification and subsequent claims to refer to specific components or parts. Those with ordinary knowledge in the field should understand that manufacturers may use different names or terminology to refer to the same component or part. This specification and the subsequent claims do not distinguish components or parts based on differences in names but rather on differences in their functions as the criterion for distinction. The terms “comprising” and “including” mentioned throughout the specification and subsequent claims are open expressions and should be interpreted as “including but not limited to”. Additionally, the term “connected” herein encompasses any direct or indirect electrical connection methods. Indirect electrical connection methods include connections through other devices.

The flexible neck massager provided by the embodiment of the utility model will be described in detail below with reference to the drawings as well as specific embodiments and application scenarios.

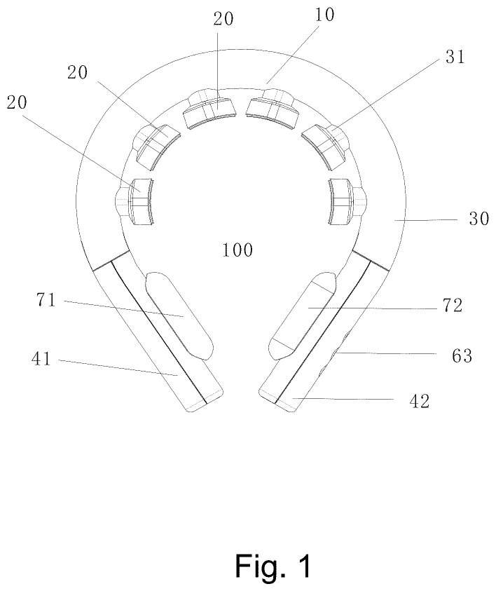

As shown in FIGS. 1-3, the first embodiment of the utility model provides a flexible neck massager 100, comprising a housing 10 and a plurality of massage heads 20, wherein the housing 10 comprises a support body 30 and two holding arms 40. The support body 30 is a flexible support body 30 made of flexible materials, at least one metal strip 80 is arranged in the flexible support body 30, and the two holding arms 40 are respectively arranged at both ends of the flexible support body 30. Preferably, the flexible material is silicone, etc. Silicone has good elasticity and can adapt to different neck shapes of the individuals, making it more comfortable to wear and enhancing the massage effect. Additionally, the metal strip 80 is arranged in the flexible support body 30 and can enhance the structural hardness of the flexible support body 30, ensuring that the flexible support body 30 has a good holding and fixing effect. Preferably, the holding arm 40 is a hard holding arm 40 made of hard materials. The two holding arms 40 are respectively arranged at both ends of the flexible support body 30. The hard material is plastic, etc. Plastic has a good holding and fixing effect.

The plurality of massage heads 20 are fixed on the inner side of the flexible support body 30. A first accommodating cavity is arranged in the flexible support body 30, a first control circuit board 51 is arranged in the first accommodating cavity, and the plurality of massage heads 20 are electrically connected to the first control circuit board 51. Preferably, the first control circuit board 51 is a flexible circuit board, which is easy to assemble, lightweight and space-saving.

The support body of the flexible neck massager is made of flexible material, which has large elasticity, thereby adapt to the neck shapes of different human bodies, making it more comfortable to wear and improving the massage effect. Additionally, the holding arms are independently designed to facilitate the assembly and disassembly of components (such as electronic components, control circuit boards, etc.) in the flexible neck massager 100. Moreover, the holding arms are preferably made of hard materials, thus having a good holding and fixing effect.

Preferably, the metal strip 80 is embedded in the flexible support body 30 during injection molding. Alternatively, the flexible support body 30 is provided with at least one accommodating channel, and the metal strip 80 is inserted into the accommodating channel.

Preferably, the metal strip is made of common metal or memory metal. The common metals include stainless steel, aluminum alloy, copper, etc. The memory metal can return to its pre-set shape after deformation and has super elasticity, making it less prone to fatigue fracture.

Preferably, the holding arm 40 is a hard holding arm 40 made of hard materials. The hard holding arm has a good holding and fixing effect. Of course, the holding arm 40 can also be made of flexible materials.

Preferably, the flexible support body 30 is provided with a first fixing groove in the first accommodating cavity adapted to the shape of the first control circuit board 51 to ensure that the first control circuit board 51 can be stably fixed in the first fixing groove.

Preferably, at least one reinforcing rib (not shown in the drawing) is arranged in the first accommodating cavity of the flexible support body 30. The reinforcing ribs are used to enhance the structural strength of the flexible support body 30.

Preferably, the flexible support body 30 is provided with at least one first opening (not shown in the drawing) at the position of the first accommodating cavity, the first opening is provided with a first sealing strip, and the first opening is detachably sealed through the first sealing strip. To install or maintain the first control circuit board 51 and/or the first electronic unit 61 in the first accommodating cavity of the flexible neck massager 100, simply lift the first sealing strip to open the first opening. After installation or maintenance is completed, close the first sealing strip to seal the first opening.

Preferably, a plurality of mounting holes 31 for mounting the plurality of massage heads 20 are provided on the inner side of the flexible support body 30.

Preferably, a first electronic unit 61 is also arranged on the flexible support body 30 or in the first accommodating cavity and electrically connected to the first control circuit board 51. Preferably, a first fixing groove adapted to the shape of the first electronic unit 61 is arranged in the first accommodating cavity of the flexible support body 30 to ensure that the first electronic unit 61 is stably fixed in the first fixing groove.

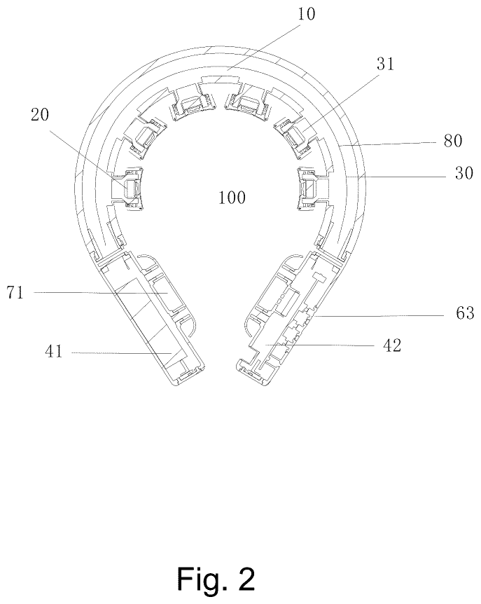

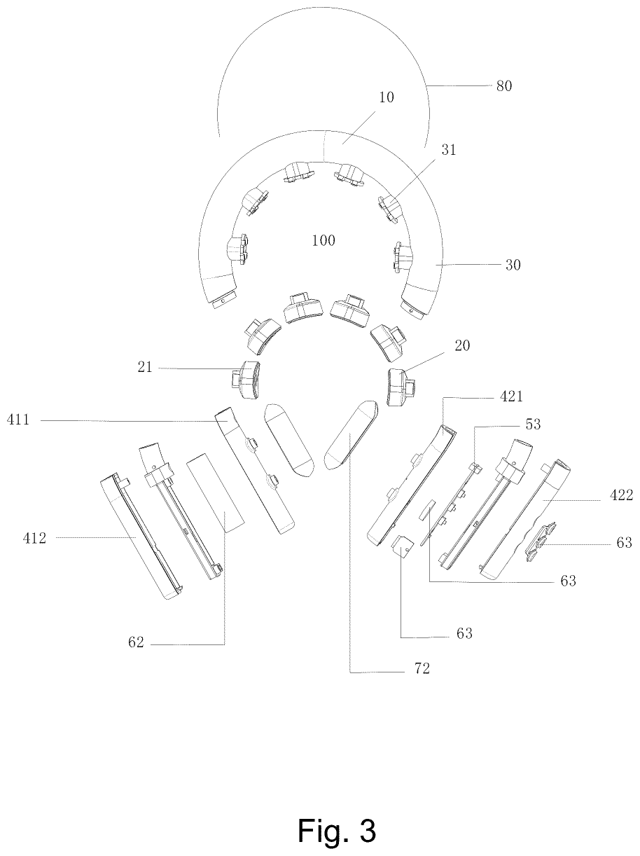

Preferably, each massage head 20 comprises a massage head housing 21 and electrodes 22; as shown in FIGS. 2-3, the massage head housing 21 is provided with a massage head accommodating cavity, and the electrodes 22 are fixed at the top of the massage head housing 21 and electrically connected to the first control circuit board 51.

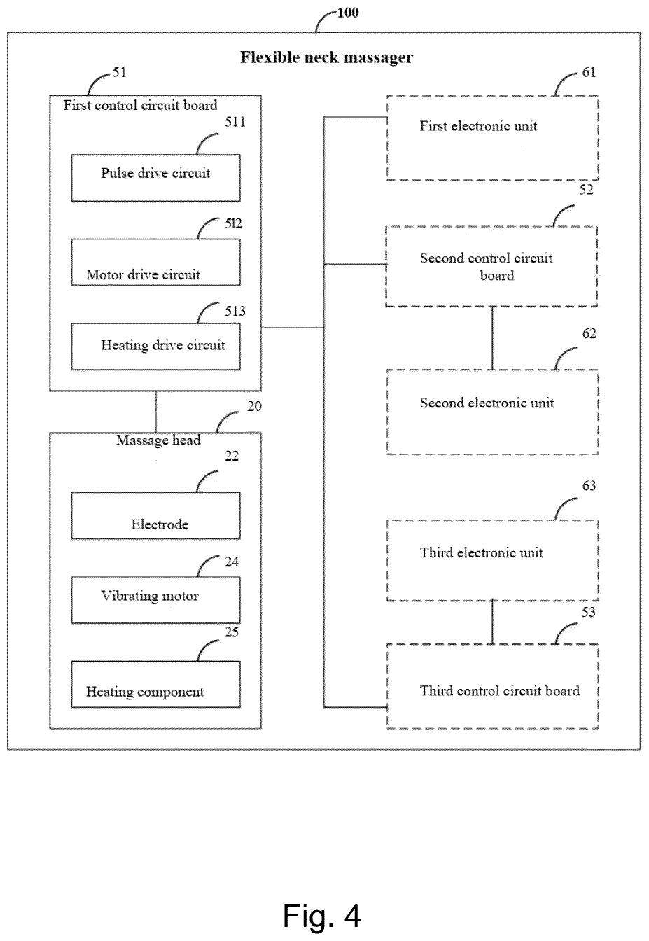

FIG. 4 is a circuit diagram of the flexible neck massager provided in Embodiment 2 of the utility model.

Preferably, the first control circuit board 51 comprises a pulse drive circuit 511, which is electrically connected to the electrodes 22 of the plurality of massage heads 20, and the electrodes 22 output pulse current according to the pulse control instructions of the pulse drive circuit 511. In this embodiment, the first control circuit board 51 controls the electrodes 22 to perform pulse massage. Preferably, the electrodes 22 are controlled to generate pulse current according to the preset frequency and/or duration. For example, the pulse frequency of the flexible neck massager 100 is divided into multiple levels. Pulse current is generated according to different levels of pulse frequency to achieve different degrees of pulse massage on the human neck. Accordingly, the pulse duration can also be controlled by control signals sent from the first control circuit board 51.

Preferably, a vibrating motor 24 is arranged in the massage head accommodating cavity of the massage head housing 21. The first control circuit board 51 comprises a motor drive circuit 512, which is electrically connected to the vibrating motors 24 of the plurality of massage heads 20, and the vibrating motors 24 vibrate according to the vibration control instructions of the motor drive circuit 512.

Preferably, the first control circuit board 51 can control the electrodes 22 and the vibrating motor 24. In other words, the flexible neck massager 100 can simultaneously perform vibration massage and pulse massage. On the one hand, the vibrating motors 24 vibrate according to the vibration control instructions of the motor drive circuit 512. On the other hand, the electrodes 22 are controlled to generate pulse current according to the preset frequency and/or duration to achieve varying degrees of pulse massage on the human neck. In this embodiment, the first control circuit board 51 controls the electrodes 22 and the vibrating motor 24 to perform pulse massage. For example, the vibrating motor 24 is controlled to vibrate according to the preset frequency and/or duration. For instance, the vibration frequency of the flexible neck massager 100 is divided into multiple levels. The vibrating motor 24 vibrates according to different levels of vibration frequency to achieve different degrees of vibration massage on the human neck. Accordingly, the massage duration can also be controlled by control signals sent from the first control circuit board 51.

Preferably, a heating component 25 is arranged in the massage head accommodating cavity of the massage head housing 21. The first control circuit board 51 comprises a heating drive circuit 513, which is electrically connected to the heating components 25 of the plurality of massage heads 20; the heating components 25 heat up according to the heating control instructions of the heating drive circuit 513. In this embodiment, the first control circuit board 51 realizes the operation control of the heating component 25, for example, controlling the heating component 25 to heat up according to the preset temperature and/or duration. For instance, the heating temperature of the flexible neck massager 100 is divided into multiple grades. Heating is carried out according to the heating temperature of different grades to achieve different temperatures for massaging the human neck. Accordingly, the heating duration can also be controlled by control signals sent from the first control circuit board 51. The heating component 25 can achieve the function of heating the human neck. The appropriate temperature allows users wearing the flexible neck massager 100 to obtain a better massage effect. The preset temperature in the utility model is 25-50 degrees C., preferably 40 degrees C., 35 degrees C., etc.

Preferably, as shown in FIGS. 1-3, the holding arm 40 comprises a first holding arm 41 and a second holding arm 42. Arc-shaped surfaces adapted to the human neck are arranged on the inner sides of the first holding arm 41 and the second holding arm 42, making users wearing the flexible neck massager 100 feel comfortable.

Preferably, a second accommodating cavity is arranged in the first holding arm 41, at least one second electronic unit 62 is arranged on the first holding arm 41 or in the second accommodating cavity, and a second control circuit board 52 is arranged in the second accommodating cavity and electrically connected to the second electronic unit 62 and the first control circuit board 51. Optionally, the second control circuit board 52 is a flexible circuit board, which is easy to assemble, lightweight and space-saving. As shown in FIGS. 1-3, the second electronic unit 62 is a battery unit 62. Of course, the second electronic unit 62 can also be a display screen, a control button, a speaker, etc. Optionally, the second control circuit board 52 is a flexible circuit board, which is easy to assemble, lightweight and space-saving. The battery unit 62 can be either a rechargeable battery or a non-rechargeable battery. It is obvious that the flexible neck massager 100 of the utility model may also not comprise the second control circuit board 52 and/or the second electronic unit 62.

Preferably, a third accommodating cavity is arranged in the second holding arm 42, at least one third electronic unit 63 is arranged on the second holding arm 42 or in the third accommodating cavity, and a third control circuit board 53 is arranged in the third accommodating cavity and electrically connected to the third electronic unit 63 and the first control circuit board 51. Optionally, the third control circuit board 53 is a flexible circuit board, which is easy to assemble, lightweight and space-saving. It is obvious that the flexible neck massager 100 of the utility model may also not comprise the third control circuit board 53 and/or the third electronic unit 63.

Preferably, in this embodiment, the third electronic unit 63 comprises a display screen 631, control buttons 632 and a charging interface 633, etc. Of course, the third electronic unit 63 can also be a speaker, a touch screen, etc. The display screen 631 is used to display the current operating status of the flexible neck massager 100. The control buttons 632 are provided for users to perform control operations, such as on/off operations, setting massage time and intensity, etc. The charging interface 633 is electrically connected to the battery unit 62, and the charging interface 633 can be connected to an external power supply to charge the battery unit 62. The battery unit 62 can also be omitted. The charging interface 633 can be directly connected to a household power supply via a wire to provide power for the flexible neck massager 100. Of course, the charging interface 633 can also be arranged on the first flexible holding arm 41 or the flexible support body 30.

Preferably, the first holding arm 41 is composed of a first holding arm inner housing 411 and a first holding arm outer housing 412. As shown in FIG. 2, the first holding arm inner housing 411 and the first holding arm outer housing 412 jointly form a second accommodating cavity. The first holding arm inner housing 411 is arranged on the inner side of the neck massager 100, and the first holding arm outer housing 412 is arranged on the outer side of the neck massager 100. Moreover, the arc-shaped surface adapted to the human neck is arranged on the inner side of the first holding arm inner housing 411, making users wearing the flexible neck massager 100 feel comfortable. Optionally, both the first holding arm inner housing 411 and the first holding arm outer housing 412 are made of plastic materials.

Preferably, the second holding arm 42 is composed of a second holding arm inner housing 421 and a second holding arm outer housing 422. As shown in FIG. 2, the second holding arm inner housing 421 and the second holding arm outer housing 422 jointly form a fourth accommodating cavity. The second holding arm inner housing 421 is arranged on the inner side of the neck massager 100, and the second holding arm outer housing 422 is arranged on the outer side of the neck massager 100. Moreover, the arc-shaped surface adapted to the human neck is arranged on the inner side of the second holding arm inner housing 421, making users wearing the flexible neck massager 100 feel comfortable. Optionally, both the second holding arm inner housing 421 and the second holding arm outer housing 422 are made of plastic materials.

Preferably, a second fixing groove adapted to the shape of the second control circuit board 52 is arranged in the second accommodating cavity of the first holding arm 41 to ensure that the second control circuit board 52 is stably fixed in the second fixing groove.

Preferably, a third fixing groove adapted to the shape of the third control circuit board 53 is arranged in the third accommodating cavity of the second holding arm 42 to ensure that the third control circuit board 53 is stably fixed in the third fixing groove.

Preferably, a first flexible structure 71 is arranged on the inner side of the first holding arm 41; and/or
a second flexible structure 72 is arranged on the inner side of the second holding arm 42. The first flexible structure 71 and/or the second flexible structure 72 are made of flexible materials such as silicone, providing a comfortable wearing experience for users wearing the neck massager 100. Preferably, the first flexible structure 71 and/or the second flexible structure 72 have decorative shapes, achieve a decorative and aesthetic effect.

To sum up, the utility model provides a flexible neck massager, which comprises a housing and a plurality of massage heads, wherein the housing comprises a flexible support body and two holding arms; the plurality of massage heads are fixed on the inner side of the flexible support body; a first accommodating cavity is arranged in the flexible support body, a first control circuit board is arranged in first accommodating cavity, and he plurality of massage heads are electrically connected to the first control circuit board. Thus, the support body of the flexible neck massager in the utility model is made of flexible materials, allowing it to adapt to the neck shapes of different individuals, providing greater comfort during wear and enhancing the massage effect. Additionally, the metal strip is arranged in the flexible support body, ensuring structural rigidity. Moreover, the independently designed holding arms facilitate the assembly and disassembly of components.

Certainly, the utility model may be embodied in various other forms. Without departing from the spirit and essence of the utility model, those skilled in the art can make corresponding modifications and variations based on the utility model. However, all such modifications and variations shall fall within the scope of protection defined by the appended claims of the utility model.

1. A flexible neck massager, comprising a housing and a plurality of massage heads, wherein the housing comprises a support body and two holding arms; the support body is a flexible support body made of flexible materials, and at least one metal strip is arranged in the flexible support body; the two holding arms are respectively arranged at both ends of the flexible support body; the plurality of massage heads are fixed on the inner side of the flexible support body; a first accommodating cavity is arranged in the flexible support body, a first control circuit board is arranged in the first accommodating cavity, and the plurality of massage heads are electrically connected to the first control circuit board.

2. The flexible neck massager according to claim 1, wherein the metal strip is embedded in the flexible support body during injection molding; or
the flexible support body is provided with at least one accommodating channel, and the metal strip is inserted into the accommodating channel.

3. The flexible neck massager according to claim 1, wherein the metal strip is made of common metal or memory metal.

4. The flexible neck massager according to claim 1, wherein the flexible support body is provided with a first fixing groove in the first accommodating cavity adapted to the shape of the first control circuit board, and the first control circuit board is fixed in the first fixing groove; and/or
the flexible support body is provided with at least one first opening at the position of the first accommodating cavity, the first opening is provided with a first sealing strip, and the first opening is detachably sealed through the first sealing strip; and/or
a plurality of mounting holes for mounting the plurality of massage heads are provided on the inner side of the flexible support body; and/or
at least one reinforcing rib is arranged in the first accommodating cavity of the flexible support body; and/or
a first electronic unit is also arranged on the flexible support body or in the first accommodating cavity and electrically connected to the first control circuit board; and/or
the holding arm is a hard holding arm made of hard materials.

5. The flexible neck massager according to claim 1, wherein each of the massage heads comprises a massage head housing and electrodes, the massage head housing is provided with a massage head accommodating cavity, and the electrodes are fixed at the top of the massage head housing and electrically connected to the first control circuit board.

6. The flexible neck massager according to claim 5, wherein the first control circuit board comprises a pulse drive circuit, which is electrically connected to the electrodes of the plurality of massage heads, and the electrodes output pulse current according to the pulse control instructions of the pulse drive circuit; and/or
a vibrating motor is arranged in the massage head accommodating cavity of the massage head housing; the first control circuit board comprises a motor drive circuit, which is electrically connected to the vibrating motors of the plurality of massage heads, and the vibrating motors vibrate according to the vibration control instructions of the motor drive circuit; and/or
a heating component is arranged in the massage head accommodating cavity of the massage head housing; the first control circuit board comprises a heating drive circuit, which is electrically connected to the heating components of the plurality of massage heads; the heating components heat up according to the heating control instructions of the heating drive circuit.

7. The flexible neck massager according to any of claims 1-6, wherein the holding arm comprises a first holding arm and a second holding arm;
a second accommodating cavity is arranged in the first holding arm, at least one second electronic unit is arranged on the first holding arm or in the second accommodating cavity, and a second control circuit board is arranged in the second accommodating cavity and electrically connected to the second electronic unit and the first control circuit board; and/or
a third accommodating cavity is arranged in the second holding arm, at least one third electronic unit is arranged on the second holding arm or in the third accommodating cavity, and a third control circuit board is arranged in the third accommodating cavity and electrically connected to the third electronic unit and the first control circuit board.

8. The flexible neck massager according to claim 7, wherein the first holding arm is composed of a first holding arm inner housing and a first holding arm outer housing;
the second holding arm is composed of a second holding arm inner housing and a second holding arm outer housing.

9. The flexible neck massager according to claim 7, wherein a second fixing groove adapted to the shape of the second control circuit board is arranged in the second accommodating cavity of the first holding arm, and the second control circuit board is fixed in the second fixing groove; and/or
a third fixing groove adapted to the shape of the third control circuit board is arranged in the third accommodating cavity of the second holding arm, and the third control circuit board is fixed in the third fixing groove.

10. The flexible neck massager according to claim 7, wherein a first flexible structure is arranged on the inner side of the first holding arm; and/or
a second flexible structure is arranged on the inner side of the second holding arm.
